(19) United States
(12) Patent Application Publication
(10) Pub. No.: US 2020/0038216 A1
(43) Pub. Date: Feb. 6, 2020

(54) Motion Supporting Leg Brace Device

(71) Applicant: Mina Hanna, Staten Island, NY (US)

(72) Inventor: Mina Hanna, Staten Island, NY (US)

(21) Appl. No.: 16/054,650

(22) Filed: Aug. 3, 2018

(51) Int. Cl. A61F 5/01

(52) U.S. Cl. CPC A61F 5/01; A61F 2005/0169; A61F 2005/0155; A61F 2005/0167; A61F 2005/0165

(57) ABSTRACT

A motion supporting leg brace device provides a leg brace adjustable to increase or decrease weight transference to the leg brace as needed and adjusting of range of motion while wearing the leg brace. The device includes an upper brace section configured for coupling to a thigh of a user and a lower brace section configured for coupling to a shin of the user. The lower brace is pivotally coupled to the upper brace section by a hinge. A sole is coupled to the lower brace section such that the upper brace section, the lower brace section, and the sole define a brace. A shock absorber is coupled between the sole and the lower brace section wherein movement of the sole towards and away from the lower brace section is dampened by the shock absorber.

### CROSS-REFERENCE TO RELATED APPLICATIONS

I hereby claim the benefit under 35 U.S.C. Section 119(e) of U.S. Provisional application 62/541,315 filed Aug. 4, 2017.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to brace devices and more particularly pertains to a new brace device for providing a leg brace adjustable to increase or decrease weight transference to the leg brace as needed and adjust range of motion while wearing the leg brace.

### BRIEF SUMMARY OF THE INVENTION

An embodiment of the disclosure meets the needs presented above by generally comprising an upper brace section configured for coupling to a thigh of a user and a lower brace section configured for coupling to a shin of the user. The lower brace is pivotally coupled to the upper brace section by a hinge. A sole is coupled to the lower brace section such that the upper brace section, the lower brace section, and the sole define a brace. A shock absorber is coupled between the sole and the lower brace section wherein movement of the sole towards and away from the lower brace section is dampened by the shock absorber.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top front side perspective view of a motion supporting leg brace device according to an embodiment of the disclosure.

FIG. 2 is a front view of an embodiment of the disclosure.

FIG. 3 is a side view of an embodiment of the disclosure.

FIG. 4 is a side view of an embodiment of the disclosure.

FIG. 5 is a top view of an embodiment of the disclosure.

FIG. 6 is a side view of an embodiment of the disclosure.

### DETAILED DESCRIPTION OF THE INVENTION

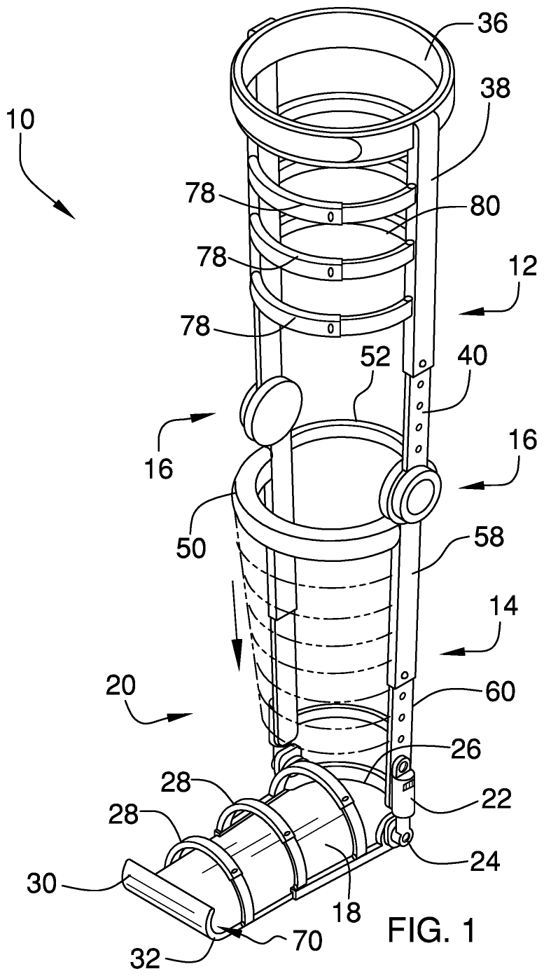

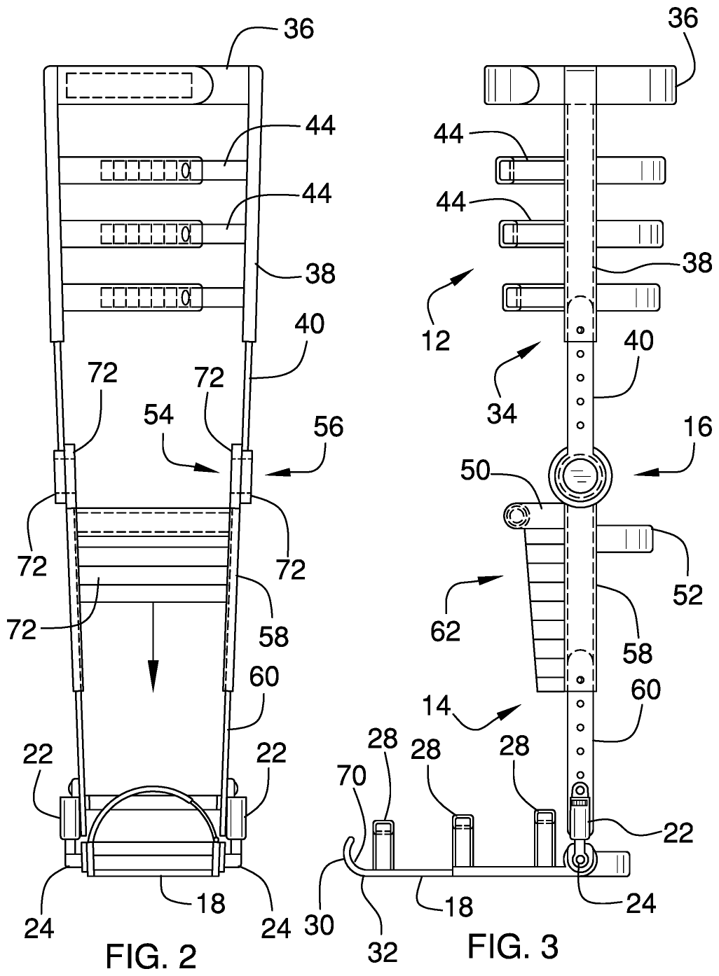

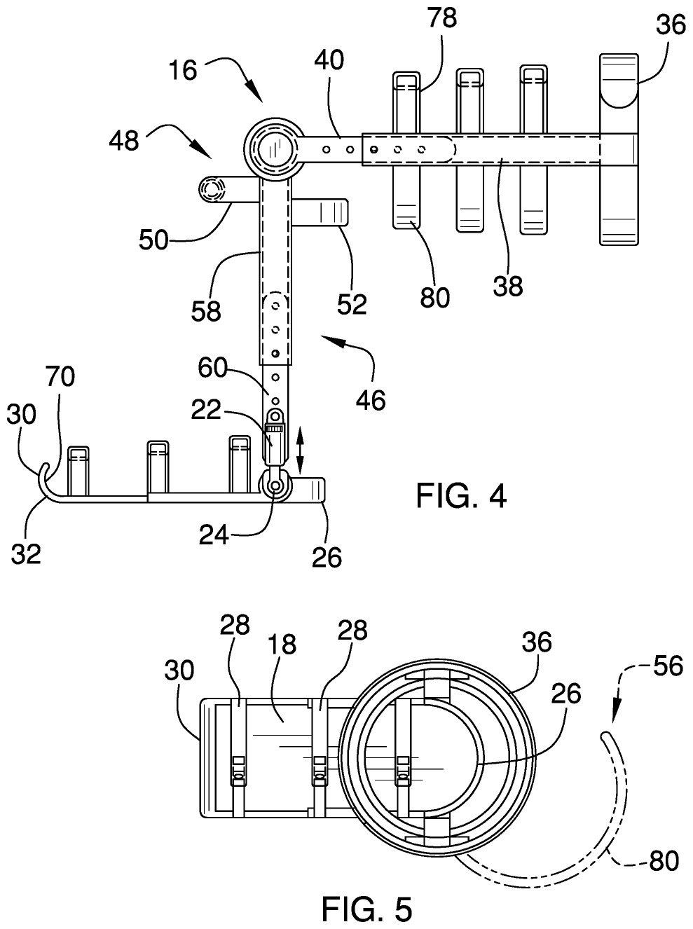

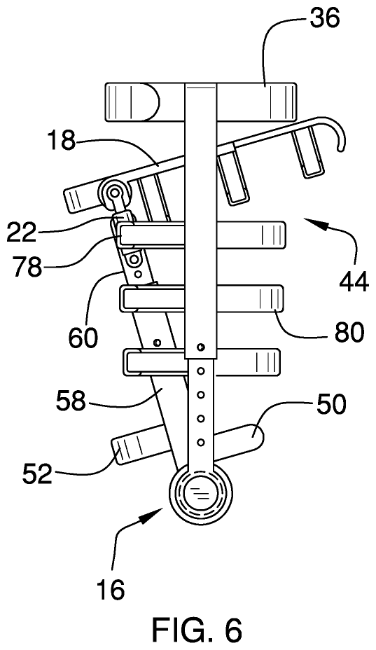

With reference now to the drawings, and in particular to FIGS. 1 through 6 thereof, a new brace device embodying the principles and concepts of an embodiment of the disclosure and generally designated by the reference numeral 10 will be described.

As best illustrated in FIGS. 1 through 6, the motion supporting leg brace device 10 generally comprises an upper brace section 12 configured for coupling to a thigh of a user. A lower brace section 14 is configured for coupling to a shin of the user. A hinge 16 couples the upper brace section 14 to the lower brace section 14 wherein the lower brace section 14 is pivotally coupled to the upper brace section 12. A sole 18 is planar and configured to be positioned under a foot of the user. The sole 18 is coupled to the lower brace section 14 wherein the upper brace section 12, the lower brace section 14, and the sole 18 define a brace 20.

A shock absorber 22 is coupled between the sole 18 and the lower brace section 14 wherein movement of the sole 18 towards and away from the lower brace section 14 is dampened by the shock absorber 22. A base end 24 of the shock absorber 22 is pivotally coupled to the sole 18. The base end 24 of the shock absorber 22 is positioned closer to a rear edge 26 of the sole 18 than a forward edge 32 of the sole 18. A range of motion of the shock absorber 22 is adjustable, in a conventional manner which would be known to those skilled in the art, for limiting movement of the sole 18 towards and away from the lower brace section 14. The shock absorber 22 is also adjustable for stiffness, using conventional methods and structures for shock absorbers, such that resistance to movement of the sole 18 towards and away from the lower brace section 14 is adjustable. Adjustment of the shock absorber 22 may be made using conventional methods including having a dial or the like positioned on the shock absorber 22 or by loosening and tightening of a nut with an adjustment tool or the like. Thus, the amount of weight borne by the brace 20, the user, or a combination thereof is adjustable.

Further, a range of motion of the hinge 16 may be restricted for keeping the lower brace section 14 substantially in alignment with the upper brace section 12 or at a desired angle such as supporting a lower section of the leg at a 45 degree angle or 90 degree angle relative to the thigh the user. This may be achieved in a conventional manner using interlocking toothed discs 72 each being coupled to an associated one of the upper brace section 12 and the lower brace section 14. A locking mechanism 74 of conventional design is provided for holding the discs in a static position relative to each other. The locking mechanism 74 may be of a type adjustable with or without a dedicated adjustment tool.

The hinge 16 may also utilize conventional and known structures for allowing rotation between the discs 72. This allows the user to resume with a more natural walking motion while still having partial to full weight bearing by the brace 20. Using conventional structures, the rotation of the discs 72 may be limited to range of angles including a maximum angle between the upper brace section 12 and the lower brace section 14 while in use to prevent hyperextension or overextension of the knee joint during use. Thus, rotation of the lower brace section 14 relative to the upper brace section 12 may be adjustable to limit a range of motion of the lower brace section 14 relative to the upper brace section 12. The range of motion of the hinge 16 may also be adjustable in a conventional fashion. Conventional structure may also be incorporated such that tension in the hinge 16 is adjustable for preventing the hinge 16 from being pivoted below a threshold amount of force is applied to the hinge 16. It is also contemplated that the hinge 16 may incorporate conventional structure to allow for full release of the discs 72 from each other when the brace 20 is not being worn to allow for folding the brace 20 into a compact position to facilitate transport or storage while not in use.

Each of a plurality of foot straps 28 is coupled to the sole 18. The foot straps 28 have opposite ends coupled to opposing lateral sides of the sole 18 wherein the sole 18 is configured for being secured onto the foot of the user by the foot straps 28. A toe guard 30 having a concave side 70 facing the foot is coupled to and may be an integral extension extending upwardly from the forward edge 32 of the sole 18. The toe guard 30 is arcuate curling up and then back towards the leg of the user.

The upper brace section 12 more specifically includes a pair of upper braces 34 and a thigh strap 36. Each of the upper braces 34 is coupled to and extends downwardly from the thigh strap 36. The thigh strap 36 is securable into a loop wherein the thigh strap 36 is configured for being extended around the thigh of the user. Thus, the upper brace section 12 is secured to the thigh of the user. The upper braces 34 are oppositionally positioned wherein the pair of upper braces 34 are configured for extending respectively down an interior side and an exterior side of the thigh of the user aligning the hinge 16 with the knee. Each upper brace 34 has a top section 38 and a bottom section 40. The bottom section 40 is coupled to the top section 38 such that a length of each upper brace 34 is adjustable. As shown alignable holes 42 may receive a pin 42 locking the top section 38 into a static position relative to the bottom section 40.

Each of a plurality of ratcheting straps 44 forms a respective ring and is coupled to the top sections 38 of the upper braces 34 such that the ratcheting straps 44 are configured to extend around the thigh of the user such that weight of the user is borne by the upper brace section 38. Each of the ratcheting straps 44 may be ratcheted in a conventional manner for tightening around and securement to the thigh. Each ratcheting strap 44 has a respective front section 78 and rear section 80. Each front section 78 and rear section 80 comprises a metal frame insert 76 enclosed by a foam rubber pad 78. The ratcheting straps 44 may include at least one of the front section 78 and the rear section 80 having a releasable hooked end 82 allowing the ratcheting strap 44 to be moved out of the way to allow the lower brace section 14 to be folded up into the upper brace section 12 for storage or transport.

The lower brace section 14 more specifically includes a pair of lower braces 46 and a knee support 48. Each of the lower braces 46 is coupled to the knee support 48. The knee support 48 has a front portion 50 extending forwardly and between the lower braces 46 and a rear portion 52 extending rearwardly and between the lower braces 46. The front portion 50 may be offset relative to the rear portion 52 but the knee support is generally configured for extending around a leg of the user under the knee of the user. The hinge 16 comprises an inner portion 54 and an outer portion 56. Each of the lower braces 46 is coupled to a respective one of the inner portion 54 and the outer portion 56 of the hinge 16 such that each lower brace 46 is coupled to and extends down from an associated one of the upper braces 34. Each lower brace 46 has an upper section 58 and a lower section 60. Similar to the top section 38 and the bottom section 40 of the upper braces 34, the lower section 60 is coupled to the upper section 58 such that a length of each lower brace 46 is adjustable. Again, this may be done using aligned holes and pins or a similar conventional mechanism.

A shield 62 is coupled to the front portion 50 of the knee support 48. The shield 62 extends downwardly from the front portion 50 of the knee support 48 and between the lower braces 46. The shield 62 is retractable and extendable relative to the front portion 50 of the knee support 48.

In use, the upper braces 34 and lower braces 46 are adjusted for length to best fit the user. The thigh strap 36 and ratcheting straps 44 secure the upper brace section 12 to the leg. The sole 18 is held in place using the foot straps 28. The brace 20 supports weight and adjustment of the shock absorber 22 and hinge 16 allow for virtually all weight to be borne by the brace 20. Over time, as the user's leg becomes stronger, the brace 20 can bear less weight until the brace is ultimately no longer needed. Adjustments can be made to allow for greater motion and for the user to bear more weight.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A motion supporting leg brace device comprising:
an upper brace section configured for coupling to a thigh of a user.
a lower brace section configured for coupling to a shin of the user.
a hinge coupling said upper brace section to said lower brace section wherein said lower brace is pivotally coupled to said upper brace section.
a sole configured to be positioned under of foot of the user, said sole being coupled to said lower brace section wherein said upper brace section, said lower brace section, and said sole define a brace. and
a shock absorber being coupled between said sole and said lower brace section wherein movement of said sole towards and away from said lower brace section is dampened by said shock.

2. The device of claim 1, further comprising a base end of said shock absorber being pivotally coupled to said sole.

3. The device of claim 2, further comprising said base end of said shock absorber being aligned with a rear edge of said sole.

4. The device of claim 1, further comprising a range of motion of said shock absorber being adjustable for limiting movement of said sole towards and away from said lower brace section.

5. The device of claim 1, further comprising said shock absorber being adjustable such that resistance to movement of said sole towards and away from said lower brace section is adjustable.

6. The device of claim 1, further comprising a range of motion of said hinge being restricted for keeping said lower brace section substantially in alignment with said upper brace section.

7. The device of claim 1, further comprising said range of motion of said hinge being adjustable.

8. The device of claim 1, further comprising tension in said hinge being adjustable for preventing said hinge from being pivoted below a threshold amount of force being applied to said hinge, rotation of said lower brace section relative to said upper brace section being adjustable to limit a range of motion of said lower brace section relative to said upper brace section.

9. The device of claim 1, further comprising a plurality of foot straps, each of said foot straps being coupled to said sole wherein said sole is configured for being secured onto the foot of the user by said foot straps.

10. The device of claim 1, further comprising said sole being planar.

11. The device of claim 10, further comprising a toe guard coupled to and extending upwardly from a forward edge of said sole.

12. The device of claim 11, further comprising said toe guard being arcuate.

13. The device of claim 1, further comprising said upper brace section including a pair of upper braces, and a thigh strap, each of said upper braces being coupled to and extending downwardly from said thigh strap, said thigh strap being securable into a loop wherein said thigh straps is configured for being extended around the thigh of the user whereby said upper brace section is secured to the thigh of the user, said upper braces being oppositionally positioned wherein said pair of upper braces are configured for extending respectively down an interior side and an exterior side of the thigh of the user.

14. The device of claim 1, further comprising each said upper brace having a top section and a bottom section, said bottom section being coupled to said top section such that a length of each said upper brace is adjustable.

15. The device of claim 14, further comprising said upper brace section including a plurality of ratcheting straps, each of said ratcheting straps forming a respective ring and being coupled to said top sections of said upper braces such that said ratcheting straps are configured to extend around the thigh of the user such that weight of the user is borne by said upper brace section.

16. The device of claim 13, further comprising said lower brace section including a pair of lower braces, and a knee support, each of said lower braces being coupled to said knee support, said knee support having a front portion extending forwardly and between said lower braces and a rear portion extending rearwardly and between said lower braces wherein said knee support is configured for extending around a leg of the user under a knee of the user, said hinge comprising an inner portion and an outer portion, each of said lower braces being pivotally coupled to a respective one of said inner portion and said outer portion of said hinge such that each said lower brace is coupled to and extends down from an associated one of said upper braces.

17. The device of claim 16, further comprising a shield coupled to said front portion of said knee support, said shield extending downwardly from said front portion of said knee support and between said lower braces.

18. The device of claim 17, further comprising said shield being retractable and extendable relative to said front portion of said knee support.

19. The device of claim 16, further comprising said lower brace section including an upper section and a lower section, said lower section being coupled to said upper section such that a length of each said lower brace is adjustable.

20. A motion supporting leg brace device comprising:
an upper brace section configured for coupling to a thigh of a user.
a lower brace section configured for coupling to a shin of the user.
a hinge coupling said upper brace section to said lower brace section wherein said lower brace is pivotally coupled to said upper brace section.
a sole, said sole being planar, said sole being configured to be positioned under of foot of the user, said sole being coupled to said lower brace section wherein said upper brace section, said lower brace section, and said sole define a brace.
a shock absorber being coupled between said sole and said lower brace section wherein movement of said sole towards and away from said lower brace section is dampened by said shock, a base end of said shock absorber being pivotally coupled to said sole, said base end of said shock absorber being aligned with a rear edge of said sole, a range of motion of said shock absorber being adjustable for limiting movement of said sole towards and away from said lower brace section, said shock absorber being adjustable such that resistance to movement of said sole towards and away from said lower brace section is adjustable.
a range of motion of said hinge being restricted for keeping said lower brace section substantially in alignment with said upper brace section, said range of motion of said hinge being adjustable, tension in said hinge being adjustable for preventing said hinge from being pivoted below a threshold amount of force being applied to said hinge, rotation of said lower brace section relative to said upper brace section being adjustable to limit a range of motion of said lower brace section relative to said upper brace section.
a plurality of foot straps, each of said foot straps being coupled to said sole wherein said sole is configured for being secured onto the foot of the user by said foot straps.
a toe guard coupled to and extending upwardly from a forward edge of said sole, said toe guard being arcuate.
wherein said upper brace section includes
a pair of upper braces, and a thigh strap, each of said upper braces being coupled to and extending downwardly from said thigh strap, said thigh strap being securable into a loop wherein said thigh straps is configured for being extended around the thigh of the user whereby said upper brace section is secured to the thigh of the user, said upper braces being oppositionally positioned wherein said pair of upper braces are configured for extending respectively down an interior side and an exterior side of the thigh of the user, each said upper brace having a top section and a bottom section, said bottom section being coupled to said top section such that a length of each said upper brace is adjustable, and
a plurality of ratcheting straps, each of said ratcheting straps forming a respective ring and being coupled to said top sections of said upper braces such that said ratcheting straps are configured to extend around the thigh of the user such that weight of the user is borne by said upper brace section, each of said ratcheting straps being padded.
wherein said lower brace section includes
a pair of lower braces, and a knee support, each of said lower braces being coupled to said knee support, said knee support having a front portion extending forwardly and between said lower braces and a rear portion extending rearwardly and between said lower braces wherein said knee support is configured for extending around a leg of the user under a knee of the user, said hinge comprising an inner portion and an outer portion, each of said lower braces being pivotally coupled to a respective one of said inner portion and said outer portion of said hinge such that each said lower brace is coupled to and extends down from an associated one of said upper braces, and
an upper section and a lower section, said lower section being coupled to said upper section such that a length of each said lower brace is adjustable. and
a shield coupled to said front portion of said knee support, said shield extending downwardly from said front portion of said knee support and between said lower braces, said shield being retractable and extendable relative to said front portion of said knee support.
